(19) United States
(12) Patent Application Publication
(10) Pub. No.: US 2015/0223964 A1
(43) Pub. Date: Aug. 13, 2015

(54) DEVICE FOR ASSISTING AND TRAINING THE HYPEREXTENSION OF PLANTAR FLEXION OF THE ANKLE AND METHOD OF USE

(71) Applicant: American Footstretcher, LLC, Flagler Beach, FL (US)

(72) Inventors: Joseph Rizzo, Flagler Beach, FL (US); Brian Mayes, Palm Coast, FL (US)

(73) Assignee: American Footstretcher, LLC, Flagler Beach, FL (US)

(21) Appl. No.: 14/178,028

(22) Filed: Feb. 11, 2014

(51) Int. Cl. A61F 5/042

(52) U.S. Cl. CPC A61F 5/042

(57) ABSTRACT

A new and useful device for assisting and training the hyperextension of plantar flexion of the ankle which can be operated by a single user, is easy to use, allows for variable adjustment of the angle of plantar flexion, is handheld, and can be used in a variety of applications and physical disciplines. This device is believed to be useful in the athletic and physical arts disciplines such as ballet. The device comprises a handle that is rotationally connected to a plantar attachment by way of a central axis rod. When connected together, the plantar attachment rotates around the central axis rod, thereby creating angles relative to the handle. The device can be locked into a desired angle by the insertion of a locking pin prior to use. The user places her foot on the sole portion of the plantar attachment, fixes an attaching means around her foot, and places her heel into a calcaneal well on the handle. Finally with the device attached to the foot, the user extends her leg and gently flexes the ankle by moving the handle of the device toward and away from the leg as needed. Over time the user's ankle will become more flexible and strong, thereby allowing proper pointe technique, alignment of the foot and minimizing injury.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### TECHNICAL FIELD

The present invention is in the technical field of mechanical stretching devices. More particularly, the present invention is in the technical field of mechanical stretching devices for use in dance and sport exercises.

### BACKGROUND OF THE INVENTION

Ballet is one of the most revered and historically significant forms of dance in the modern world. It requires of its performers a distinct combination of athleticism, elegance, discipline and artistic expression. While performing ballet, the ballerina creates the illusion of a graceful and lengthened leg by performing en pointe—a technique wherein the dancer stands on her toes, and places her entire weight on the tips of her fully extended feet. Dancing en pointe requires complete plantar flexion of the foot and ankle, with the instep fully stretched and the toes perpendicular to the floor. This technique is assisted by the use of pointe shoes that have been designed to spread the weight and stress across the foot and toe box of the shoe.

When in pointe position, the ankle is stable with the posterior lip of the tibia locked onto the calcaneus or heel bone. The subtalar joint of the foot is locked with the heel and forefoot in varus. Therefore, injury is unlikely when practiced correctly. However, injury can occur while the novice ballerina is learning and yet unable to fully extend the feet forward with full plantar flexion.

In the applicant's experience there is a deficiency in the existing and prior art wherein there are no devices for assisting and training the hyperextension of the ankle. Current techniques to assist the ballerina's progress towards a stable en pointe position with full plantar flexion include the use of bands, repeated and extensive exercises like demi-pointe techniques, bar work and manipulation and stretching of the ankle with the hand. These techniques in the current art take too long to achieve the desired result, are not all specifically targeted toward the specific joint action required for en point position, and could all benefit from a mechanical device designed to assist in the process of strengthening and stretching the ankle. A mechanical device designed to help a ballerina increase her range of plantar flexion, stretch and strengthen the ankle, and achieve a stable full en pointe position more quickly would greatly improve the art by minimizing potentially injury-prone intermediate postures.

In the applicant's experience, there is a need for a device for assisting and training the hyperextension of the ankle which i) can be operated by a single user, ii) is easy to use, ii) allows for variable adjustment of the angle of plantar flexion, iii) is handheld, and iv) can be used in a variety of applications and physical disciplines. The device of the present invention is believed to accomplish all of the foregoing objectives.

### SUMMARY OF THE INVENTION

The present invention provides a device for assisting and training the hyperextension of plantar flexion of the ankle which can be operated by a single user, is easy to use, allows for variable adjustment of the angle of plantar flexion, is handheld, and can be used in a variety of applications and physical disciplines. This device is believed to be useful in the athletic and physical arts disciplines such as ballet.

In one of its basic embodiments, the present invention comprises a handle that is rotationally connected to a plantar attachment by way of a central axis rod. The handle has a grip area near the end of the device that is closest or proximal to the user's hand when in use. At the distal end of the handle there is a receiving slot for receipt of a semicircular protrusion that is part of the plantar attachment. The plantar attachment connects to the handle of the device by means of a central axis rod that passes through the exterior central axis bore of the handle and the interior central axis bore of the plantar attachment—acting as an axle and forming a hinge.

When connected together, the plantar attachment rotates around the central axis rod, thereby creating an angle relative to the handle. The device is then locked into the desired angle by the insertion of a locking pin through the exterior fenestrations of the handle which are now aligned with the interior fenestrations of the semicircular protrusion of the plantar attachment.

The user then uses the device to stretch and strengthen her ankle, by placing her foot on the sole portion of the plantar attachment and fixing an attaching means around her foot. The attaching means can be a rubber band, strap or hook and loop flap that firmly holds the user's foot to the device when in use. Further, the user places her heel into a calcaneal well that is located near the distal end of the handle. Finally with the device attached to the foot, the user extends her leg and gently flexes the ankle by moving the handle of the device toward and away from the leg as needed. As the device moves towards the leg of the user, the ankle will experience a stretch and move towards a fully aligned and straight en point position. Over time the user's ankle will become more flexible and strong, thereby allowing proper pointe technique, alignment of the foot and minimizing injury.

Thus the present invention is believed to provide a new and useful device which can be operated by a single user, is easy to use, allows for variable adjustment of the angle of plantar flexion, is handheld, and can be used in a variety of applications and physical disciplines. Typical situations and settings that would benefit from the availability of a device for the improvement of plantar flexion of the ankle would be: ballet and dance studios, gymnastics gymnasiums, and the like.

Further features and objectives of the present invention will become apparent form the following detailed description and the accompanying drawings.

### BRIEF DESCRIPTION OF THE DRAWINGS

FIG. 1 is an exploded view of a device for assisting and training the hyperextension of plantar flexion of the ankle according to the present invention;

FIG. 2 is a perspective view of the present invention;

FIG. 3 is a front elevation view of the present invention;

FIG. 4 is a rear elevation view of the present invention;

FIG. 5 is a right side elevation view of the present invention;

FIG. 6 is a left side elevation view of the present invention with hook and loop flap attaching means unfastened and open; and

FIG. 7 is a perspective view of the present invention in its environment and on a user's leg when in use.

### DETAILED DESCRIPTION OF THE INVENTION

As described above, the present invention provides a new and useful device for assisting and training the hyperextension of plantar flexion of the ankle which can be operated by a single user, is easy to use, allows for variable adjustment of the angle of plantar flexion, is handheld, and can be used in a variety of applications and physical disciplines. This device is believed to be useful in dance studios such as a ballet school, where the dancers seek to increase the range of plantar flexion of the ankle as part of their practice and discipline. A device according to the invention is also believed to be favorable for use in athletic disciplines and even some forms of physical therapy. The following description and accompanying drawings disclose at least one version of such a device.

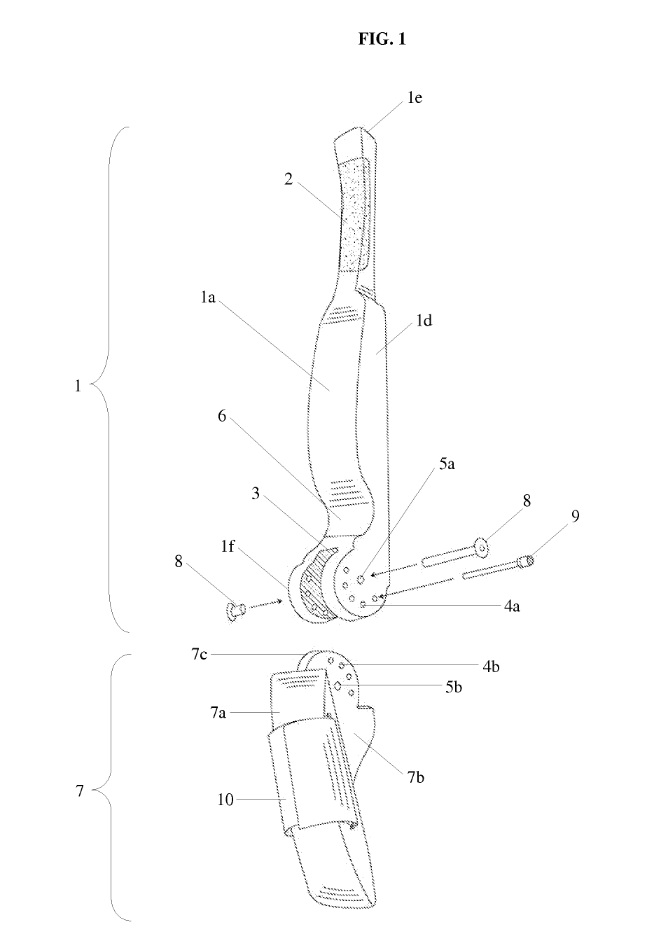

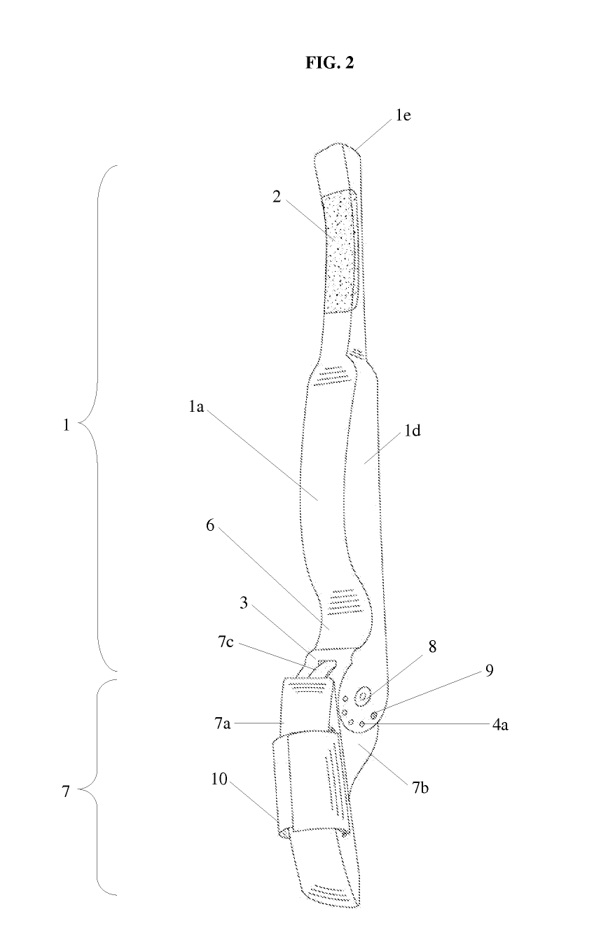

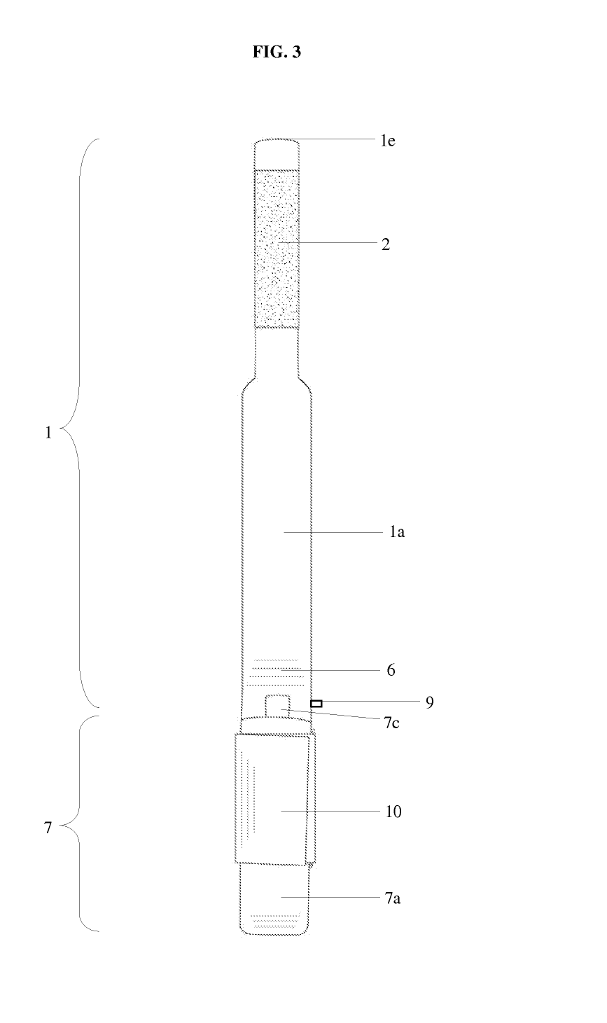

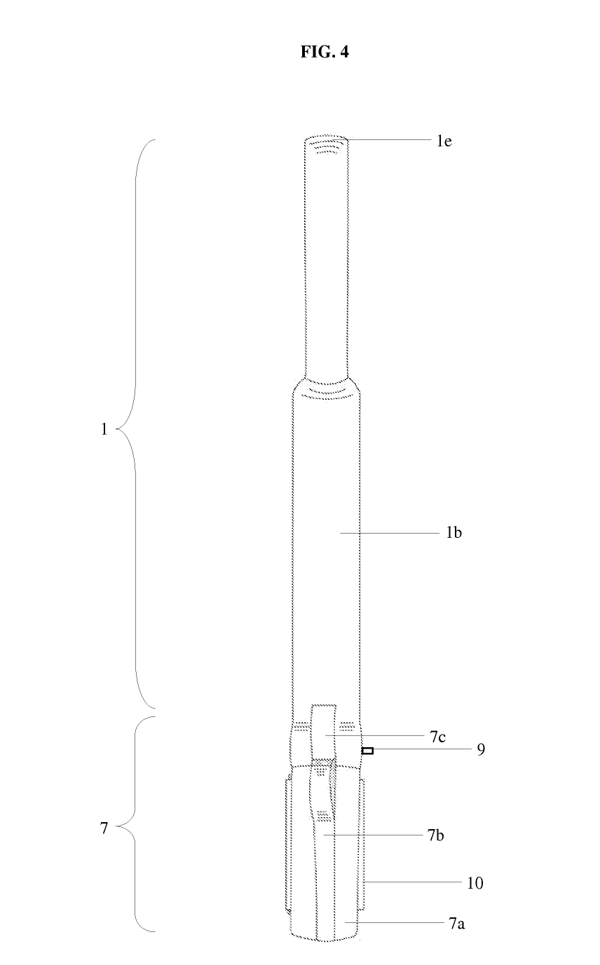

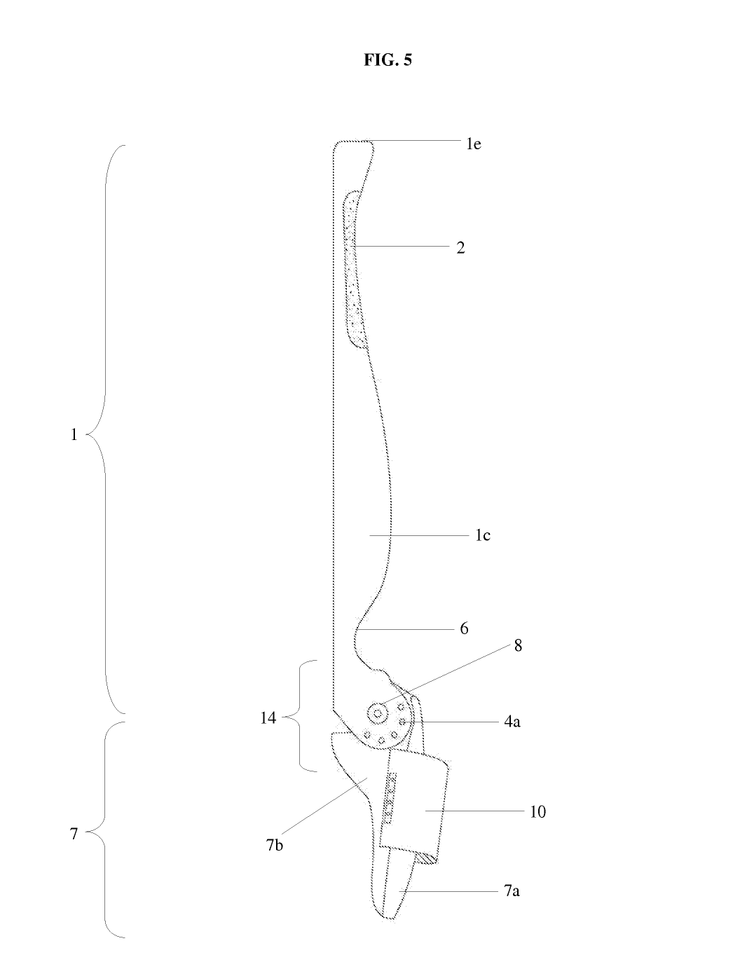

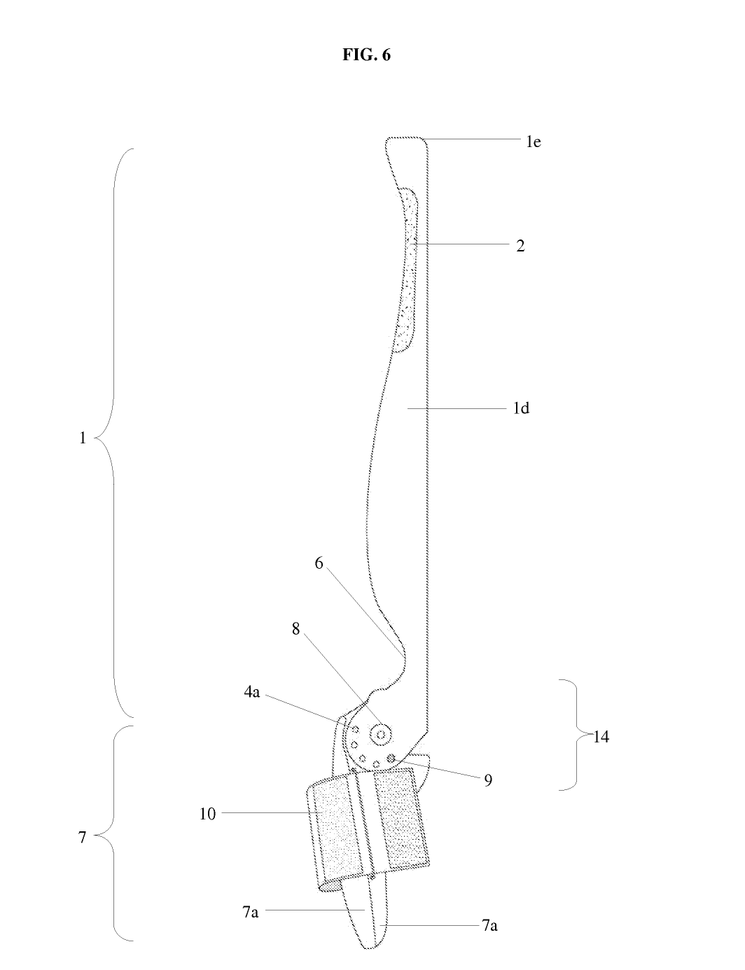

Referring now to the invention in more detail, in FIG. 1 to FIG. 7 there is shown a device for assisting and training the hyperextension of plantar flexion of the ankle of a user. It is envisioned that the user of the device is a dancer, most likely a ballerina, or other athlete. The device essentially comprises a handle 1 rotationally engaged with a plantar attachment 7. The handle 1 of the device has a front surface 1a, back surface 1b, left surface 1c and right surface 1d as well as a proximal end 1e and a distal end 1f wherein the proximal end 1e is narrower than the distal end 1f. The said distal end 1f having a generally cylindrical shape and exhibiting an essentially circular cross section. The handle 1 of the device also has a grip area 2 along the proximal end 1e of the handle 1 for receipt of a user's hand 11 when in use. A receiving slot 3 is located in the center of the distal end 1f of the handle 1, as well as a plurality of exterior fenestrations 4a at the distal end 1f of the handle 1 which are arranged in a generally semicircular configuration. An exterior central axis bore 5a is located at the center of the circular cross-section of the distal end 1f of the handle 1. A calcaneal well 6 located near the distal end 1f of the handle 1 for receipt of a user's heel 12 when the device is in use. Said calcaneal well 6 is essentially a semicircular depression near the distal end 1f of the handle 1 and along the front surface 1a of the handle 1 which is the approximate size of the human heel.

In more detail, still referring to the invention of FIG. 1 to FIG. 7, the device also has a plantar attachment 7 inserted into the receiving slot 3 to the handle 1 of the device. The plantar attachment 7 having a foot sole 7a which connects with the plantar attachment 7 of the user's foot 13 when the device is in use. Further, the plantar attachment 7 also has a plantar support structure 7b affixed perpendicularly to the underside of the foot sole 7a and having a generally semicircular protrusion 7c for engaging the plantar attachment 7 to the handle 1 of the device by insertion of the semicircular protrusion 7c of the plantar attachment 7 into the receiving slot 3 of the distal end 1f of the handle 1 of the device. A plurality of interior fenestrations 5b are also present at the distal end 1f of the handle 1 which are arranged in a generally semicircular configuration. An interior central axis bore 5b is located at the center of the semicircular protrusion 7c of the planar attachment 7.

In order to adjoin the handle 1 of the device to the plantar attachment 7, a central axis rod 8 is inserted through the exterior central axis bore 5a of the handle which is aligned with the interior central axis bore 5b of the plantar attachment 7 that is inserted into the receiving slot 3 of the handle 1 thereby attaching the plantar attachment 7 to the handle 1 when assembled. This creates a hinge 14 which allows for rotational movement of the plantar attachment 7 along the central axis rod 8 when in use. A locking pin 9 is then optionally inserted through the exterior fenestrations 4a of the distal end 1f of the handle 1 and the aligned interior fenestrations 4b of the plantar attachment 7 thereby rigidly locking the device at various angles as desired by the user when in use. The device also has an attaching means 10 which is affixed to and extending over the foot sole 7a of the plantar attachment 7 for variably affixing and tightening the device to the user's foot 13 when in use. The attaching means 10 can be a rubber band, belt, strap or hook and loop adjustable flap.

In an alternate embodiment of the present invention, the grip area 2 is covered in a slip resistant material such as rubber or foam.

In an alternate embodiment of the present invention, a cushion is placed in the calcaneal well 6 to provide for comfort and padding for the user's heel 12 when the device is in use.

In an alternate embodiment of the present invention, a cushion covers the foot sole 7a of the plantar attachment 7.

In an alternate embodiment of the present invention, a chain connects the locking pin 9 to the handle 1 of the device to prevent loss of the locking pin 9.

In an alternate embodiment of the present invention the surfaces of the device are covered with an ornamental design, which can be a design, pattern, color or text.

The dimensions of the handle 1 and plantar attachment 7 can be any size, but is preferable that the dimensions be relative to the body size of intended user. For example, an embodiment of the device which is intended to be used by child aged ballerina students would have a foot sole 7a commensurate in size to the typical foot size of a child aged ballerina student. Likewise, the dimensions of the handle 1 would be commensurate in size to the typical length of the lower leg of the intended student and allow the user hand 11 to reach the grip area 2 of the handle 1 comfortably.

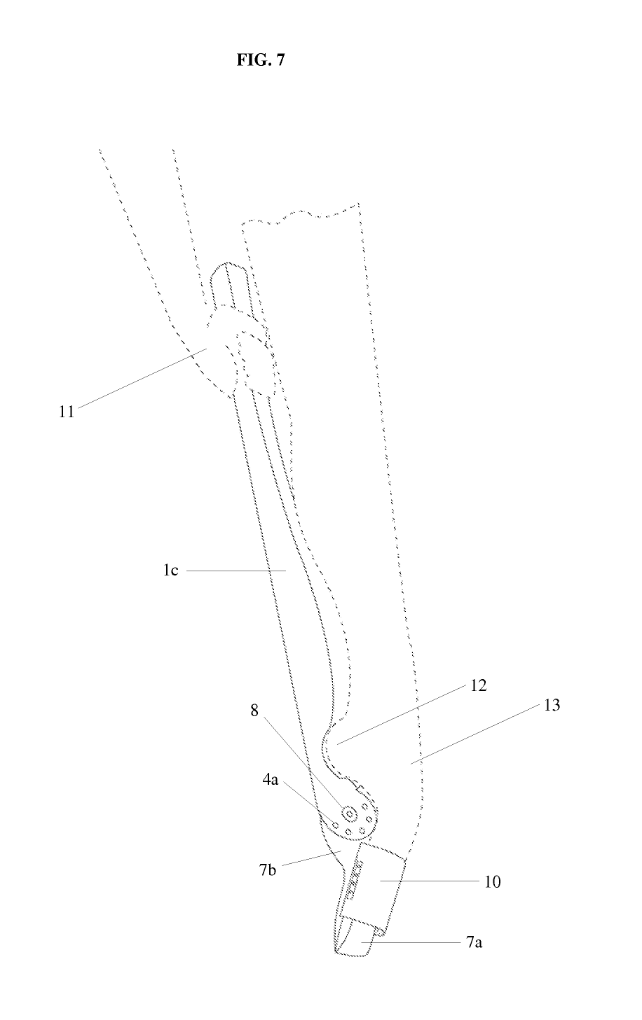

Now referring to FIG. 7 in more detail, To increase the angle of plantar flexion of a user's ankle using the device for assisting and training the hyperextension of plantar flexion of the ankle, the user begins by grabbing the device by firmly holding the grip area 2 of the proximal end 1e of the device in the user's hand 11 with the front surface 1a of the device facing upward. Then the user rotates the plantar attachment 7 with the user's other hand along the central axis of the hinge 14 created by the adjoined plantar attachment 7, handle 1, the interior central axis bore 4b and aligned exterior central axis bore 5a of the device. Next the user inserts the locking pin 9 through the exterior fenestrations 4a of the device and into the interior fenestrations 5b of the semicircular protrusion 7c of the plantar attachment 7 now inserted into the receiving slot 3 of the distal end 1f of the handle 1. By doing so, the locking pin 9 rigidly affixes the device into a configuration that creates an angle between the plantar attachment 7 and handle 1. The angle correlates to the amount of hyperextension and plantar flexion desired by the user. As the user's ankle becomes more flexible the plantar attachment 7 can be locked in more and more obtuse angles relative to the handle 1 until reaching 180 degrees or more, where the foot sole 7a of the plantar attachment 7 is essentially oriented in a straight line with the front surface 1a of the handle 1.

Next, the user holds the now rigid device with the plantar attachment 7 extended towards and near the user's foot 13. The user places her foot onto the foot sole 7a of the plantar attachment 7 and the user's heel 12 of the user's foot 13 into the calcaneal well 6 of the handle 1. The user then adjusts the attaching means 10 around and over the user's foot 13 in order to affix the user's foot 13 firmly to the device. When the user's foot 13 is firmly affixed to the device, the user extends her leg outward, while the remainder of the users body is in a stationary and comfortable posture which can be seated or standing.

The user flexes the handle 1 of the device toward the back of the leg of the user, thereby decreasing the angle formed between the handle of the device and the user's leg. This causes a very controlled and gentle forward stretching and hyperextension of the ankle and promotes further plantar flexion of the user's ankle 16. Said flexing can be varied in duration as indicated by the desired result, sport or skill. When the stretching is complete, the user removes the device from the users foot 13. The user can repeat the process over a protracted period of time as needed.

The previously described versions of the present invention have many advantages, including and without limitation, the properties of being able to be operated by a single user, easy to use, allowing for variable adjustment of the angle of plantar flexion, handheld, and used in a variety of applications and physical disciplines. The device of the present invention is believed to accomplish all of the foregoing objectives. The invention does not require that all the advantageous features and all the advantages need to be incorporated into every embodiment of the invention.

Although the present invention has been described in considerable detail with reference to certain preferred versions thereof, other versions are possible. Therefore the spirit and scope of the appended claims should not be limited to the description of the preferred versions contained therein.

The reader's attention is directed to all papers and documents which are filed concurrently with this specification and which are open to public inspection with this specification, and the contents of all such papers and documents are incorporated herein by reference.

All the features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. As for “means for” elements, the applicant intends to encompass within the language any structure presently existing or developed in the future that performs the same function. The invention should therefore not be limited by the above described embodiment, method, and examples, but by all embodiments and methods within the scope and spirit of the invention.

What is claimed is:

1. A device for assisting and training the hyperextension of plantar flexion of the ankle which comprises
a. a handle having a front surface, back surface, left surface and right surface as well as a proximal end and a distal end wherein the proximal end is narrower than the distal end, said distal end having a generally cylindrical shape and exhibiting an essentially circular cross section;
b. a grip area along the proximal end of the handle for receipt of a user's hand when in use;
c. a receiving slot located in the center of the distal end of the handle;
d. a plurality of exterior fenestrations at the distal end of the handle which are arranged in a generally semicircular configuration;
e. an exterior central axis bore at the center of the circular cross-section of the distal end of the handle;
f. a calcaneal well located near the distal end of the handle for receipt of a user's heel when in use,
said calcaneal well is essentially a semicircular depression near the distal end of the handle and along the front surface of the handle which is the approximate size of the human heel;
g. a plantar attachment inserted into the receiving slot to the handle of the device,
said plantar attachment having a foot sole portion, which connects with the plantar portion of the user's foot when the device is in use,
said plantar attachment further having a plantar support structure affixed perpendicularly to the underside of the foot sole portion and having a generally semicircular protrusion for engaging the plantar attachment to the handle of the device by insertion of the semicircular protrusion of the plantar attachment into the receiving slot of the distal end of the handle of the device;
h. a plurality of interior fenestrations at the distal end of the handle which are arranged in a generally semicircular configuration;
i. an interior central axis bore at the center of the semicircular protrusion of the planar attachment;
j. a central axis rod inserted through the external central axis bore of the handle which is aligned with the interior central axis bore of the plantar attachment that is inserted into the receiving slot of the handle thereby attaching the plantar attachment to the handle when assembled, creating a hinge which allows for rotational movement of the plantar attachment along the central axis rod when in use;
k. a locking pin, optionally inserted through the exterior fenestrations of the distal end of the handle and the aligned interior fenestrations of the plantar attachment thereby rigidly locking the device at various angles as desired by the user when in use; and
l. an attaching means affixed to and extending over the foot sole of the plantar attachment for variably affixing and tightening the device to the user's foot when in use.

2. The device as in claim 1, wherein the user is a dancer.

3. The device as in claim 1, wherein the user is an athlete.

4. The device as in claim 1, wherein the attaching means is a rubber band, belt, strap or hook and loop adjustable flap.

5. The device as in claim 1, wherein the grip area is covered in a slip resistant material such as rubber or foam.

6. The device as in claim 1, wherein the handle and plantar attachment of the device are made from a strong and rigid material such as wood, plastic or metal.

7. The device as in claim 1, further comprising a cushion in the calcaneal well.

8. The device as in claim 1, further comprising a cushion covering the foot sole of the plantar attachment.

9. The device as in claim 1, further comprising a chain connecting the locking pin to the handle of the device to prevent loss of the locking pin.

10. The device as in claim 1, further comprising an ornamental element on the surfaces of the device, which can include a design, pattern, color or other information.

11. A method for increasing the angle of plantar flexion of a user's ankle using the device for assisting and training the hyperextension of plantar flexion of the ankle which comprises
a. grabbing the device by firmly holding the grip area of the proximal end of the device in the user's hand with the front surface of the device facing upward;
b. rotating the plantar attachment with the user's other hand along the central axis of the hinge created by the adjoined plantar attachment, handle and central axis bore of the device;
c. inserting the locking pin through the external fenestrations of the device and into the interior fenestrations of the semicircular protrusion of the plantar attachment now inserted into the receiving slot of the distal end of the handle,
said locking pin thereby rigidly affixing the device into a configuration that creates an angle between the plantar attachment and handle,
said angle correlating to the amount of hyperextension and plantar flexion desired by the user;
d. holding the now rigid device with the plantar portion extended towards the foot of the user;
e. placing the foot onto the foot sole portion of the plantar attachment and the heel of the foot into the calcaneal well of the handle;
f. adjusting the attaching means around and over the foot in order to affix the foot firmly to the device;
g. extending the leg of the user outward, while the remainder of the users body is in a stationary and comfortable posture which can be seated or standing;
h. flexing the handle of the device toward the back of the leg of the user, thereby decreasing the angle formed between the handle of the device and the users leg, and gently stretching, hyperextending and promoting further plantar flexion of the users ankle,
said flexing can be varied in duration as indicated by the desired result; and
i. removing the device from the users foot when finished.

12. The method as in claim 11, further comprising repeating the steps as needed over a protracted period of time, which may be days, months or years.
